(19) United States
(12) Patent Application Publication
(10) Pub. No.: US 2022/0096234 A1
(43) Pub. Date: Mar. 31, 2022

(54) Anchored Leaflet Device And Methods For Transcatheter Valve Repair

(71) Applicant: Orsus, LLC, Plymouth, MN (US)

(72) Inventor: Paul Sorajja, Plymouth, MN (US)

(73) Assignee: Orsus, LLC, Plymouth, MN (US)

(21) Appl. No.: 17/428,601

(22) PCT Filed: Feb. 5, 2020
(86) PCT No.: PCT/US2020/016878

§ 371 Date: Aug. 4, 2021

Related U.S. Application Data
(60) Provisional application No. 62/918,561, filed on Feb. 6, 2019.

(51) Int. Cl. A61F 2/24

(52) U.S. Cl. CPC A61F 2/2454; A61F 2/2463; A61F 2/2466

(57) ABSTRACT

A leaflet repair device for a heart valve of a human heart comprises an implantable leaflet with a coaptation edge; a repair chord connected at one end to near the coaptation edge; a chord anchor for anchoring the repair chord to native structure of the human heart; and at least one annulus anchor for anchoring the leaflet to native structure of the human heart. In at least one embodiment, the leaflet repair device may overlap a diseased, prolapsed, or otherwise inferior leaflet that is not creating proper coaptation with a mating leaflet.

### RELATED APPLICATIONS

This application claims priority to U.S. Provisional Application Ser. No. 62/918,561, filed Feb. 6, 2019, entitled Anchored Leaflet Device For Transcatheter Valve Repair, which is hereby incorporated herein by reference in its entirety.

### FIELD OF THE INVENTION

The present invention relates to transcatheter-delivered valve repair and particularly to transcatheter-delivered repair of valve regurgitation.

### BACKGROUND OF THE INVENTION

Due to costs, patient preparation time, surgical time, patient recovery time, and the invasiveness of open heart surgery procedures, transcatheter-delivered devices and other minimally invasive devices provide an alternative approach to the treatment of those heart conditions that require the repair or replacement of a heart valve. One such heart condition is heart valve regurgitation, a non-limiting example of which is mitral valve regurgitation, which is commonly referred to as MR but is also referred to as mitral regurgitation, mitral insufficiency or mitral incompetence.

Mitral regurgitation (MR) is a heart condition in which the patient's mitral valve is unable to fully close which thus allows blood to abnormally flow back into the left atrium. This condition, if left untreated, often leads to heart failure.

A mitral valve typically has two leaflets, namely, a posterior leaflet and an anterior leaflet. Each leaflet is connected to the mitral annulus between the left atrial chamber and the left ventricle. When the valve is in an open position, the posterior leaflet and anterior leaflet separate to create a mitral opening that allows blood to flow from the left atrial chamber into the left ventricle. In a healthy mitral valve, when the valve is in a closed position, a coaptation surface of the posterior leaflet abuts a coaptation surface of the anterior leaflet to close the mitral opening. In a diseased or aging mitral valve, however, one or more of the leaflets may have structural deficiencies that prevent the leaflet's coaptation surface from fully abutting the coaptation surface of the other leaflet to close the mitral opening. This creates a gap between the two leaflets that allows blood to flow abnormally back into the left atrial chamber. This is mitral valve regurgitation.

Tricuspid regurgitation (TR) is a similar condition to MR but is found in the tricuspid valve. TR is a condition in which the leaflets of the tricuspid valve do not fully close, leading to abnormal flow of blood (i.e., regurgitation) back into the right atrium and the surrounding venous structures (e.g. inferior vena cava or IVC). As with MR, this condition also leads to heart failure and impaired survival. More specifically, TR arises because of inadequate leaflet apposition between two or three of the tricuspid valve leaflets (i.e., the anterior, posterior and septal leaflets). In most cases of TR, the coaptation deficiency is between the anterior and septal leaflets, with regurgitation between the posterior and septal leaflets also being frequent.

For both MR and TR, previous transcatheter repair devices have comprised annuloplasty, leaflet apposition, cordal placement, clip devices or a functional replacement valve deployed within an expandable frame. Clip devices or leaflet apposition therapies attempt to close the gap between the leaflets by spanning the distance between the leaflets that are not coapting properly. Clip devices or leaflet apposition therapies permanently affix the leaflets during both diastole and systole; limitations of these approaches include the potential for mitral stenosis and inability to replace the mitral valve without cutting of the native leaflets. Annuloplasty bands or rings, while they are used in nearly all surgical repairs, are ineffective as stand alone devices for treatment in MR in the most patients. Functional replacement valves completely relieve MR but carry risks commonly associated with prostheses, such as thrombosis, infection, and degeneration, as well as the requirement for surgical placement. The use of artificial cords can be used to treat degenerative disease, where the cord reduces leaflet height and restores coaptation, but such cords cannot be used in functional regurgitation, rheumatic disease, or other pathological conditions in which leaflet mobility is restricted.

Fully functional replacement valves circumvent the coaptation issue by deploying a device that replaces the valve. However, if and when these replacement valves fail or there is additional disease or calcification in the valve, the patient must either have a new valve inserted within the existing replacement valve or the replacement valve fully removed and a new valve inserted. Moreover, for many patients, open surgery to correct the problem is not viable leaving transcatheter repair as the sole option.

Therefore, it is desirable to improve and overcome the difficulties of previous therapies that address valve regurgitation.

### OBJECTS AND SUMMARY OF THE INVENTION

In light of the foregoing, it is an object of the present invention to provide a method and device for transcatheter-delivered treatment of valve regurgitation that improves and addresses the disadvantages of prior therapies.

It is a further object of the present invention to provide a system that is easily adaptable to a wide patent population.

It is a further object of the present invention to provide a system that minimizes the amount of structure implanted into a patient.

It is a further object of the present invention to provide a method of treating valve regurgitation that is easily practiced by medical professionals.

In this regard, the present invention is directed to systems and methods for repairing a valve, such as the mitral valve or the tricuspid valve, which includes a leaflet repair device for a heart valve of a human heart that has an implantable leaflet with a coaptation edge; a repair chord connected at one end to near the coaptation edge; a chord anchor for anchoring the repair chord to native structure of the human heart; and at least one annulus anchor for anchoring the leaflet to native structure of the human heart. In at least one embodiment, the device comprises two or more annulus, myocardial, or epicardial anchors. In at least one embodiment, the implantable leaflet comprises a tissue material. In at least one embodiment, the tissue material comprises a cross-linked, calcification resistant implantable biomaterial.

In some embodiments, a method for repairing a heart valve comprises delivering a repair chord anchor to a first location; delivering at least one annulus chord anchor to a second location substantially near a valve annulus; deploying an implantable leaflet; pushing the implantable leaflet against the annulus anchor with a pusher; and delivering an anchoring eyelet to the annulus anchor.

In some embodiments, the method further comprises pushing the implantable leaflet with the pusher towards the repair chord anchor. In some embodiments, the method further comprises delivering an anchoring eyelet to the repair chord anchor. In some embodiments, the chord may be short, used in conjunction with or replaced by structure similar to the function of the native papillary muscle. The implantable leaflet may comprise a tissue or synthetic material. The tissue material may comprise a cross-linked, calcification resistant implantable biomaterial.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1 is a cross-sectional view of a preferred embodiment of the present invention;

FIG. 2 is a cross-sectional view of a preferred embodiment of the present invention

FIGS. 3A and 3B are cross-sectional views of deployed preferred embodiments of the present invention;

FIG. 4 is a cross-sectional view of deployment of a preferred embodiment of the present invention;

FIGS. 5A-5B are cross-sectional views of a preferred embodiment of the present invention with a locking mechanism;

FIGS. 6A-6J are a series of views depicting a deployment embodiment of the present invention;

FIG. 7A is a cross sectional view of a mitral valve with regurgitation;

FIG. 7B is a cross-sectional view of mitral valve with an embodiment of the present invention;

FIGS. 8A-8B are a cross-sectional view of a tricuspid valve affected with tricuspid regurgitation in diastole;

FIGS. 9A-9B are a cross-sectional view of a tricuspid valve affected with tricuspid regurgitation in systole.

FIGS. 10A-10B are cross-sectional views of tricuspid valve in short axis affected with tricuspid regurgitation in diastole and systole.

FIGS. 11A-11B are cross-sectional views of deployment of an embodiment of the present invention in a tricuspid valve in diastole;

FIGS. 12A-12B are cross-sectional views of deployment of an embodiment of the present invention in a tricuspid valve in systole.

FIGS. 13A-13B are short axis views of a deployed embodiment of the present invention in the tricuspid valve in diastole and systole, respectively.

FIG. 14 is a cross-sectional view of a preferred embodiment of the present invention;

FIG. 15 is a cross-sectional view of a preferred embodiment of the present invention;

FIG. 16 is a cross-sectional view of a preferred embodiment of the present invention.

### DETAILED DESCRIPTION

The present invention relates to repair of heart valves using devices deployed via a catheter. Although this detailed description discusses the embodiments herein with respect to a patient's mitral heart valve or tricuspid valve, the present invention is applicable to any valve of the patient's heart and the disclosure herein should be construed as such. To be clear, the present invention as exemplified in the embodiments described herein may be applicable to the repair of other valves of the human heart.

When deployed in the patient's heart valve, the device of the present invention creates a new coaptation surface for mating with the other native leaflet and also allows for rebuilt anchoring of the leaflet to improve systolic and diastolic activity of the valve. From the perspective of the operator, the device may be echo guided with real time assessment, and deployment of the device may be reversible. The device also provides the ability to customize the leaflet to the patient based on their individual anatomy. The device of the present disclosure may be suitable for both primary and secondary causes of regurgitation as well as traumatic etiologies. Importantly, the devices of the present disclosure do not include a stent or other expandable frame.

In the following detailed description, numerous specific details are set forth in order to provide a thorough understanding of preferred embodiments. However, it will be understood by persons of ordinary skill in the art that some embodiments may be practiced without these specific details. In other instances, well-known methods, procedures, components, units, etc. have not been described in detail so as not to obscure the discussion.

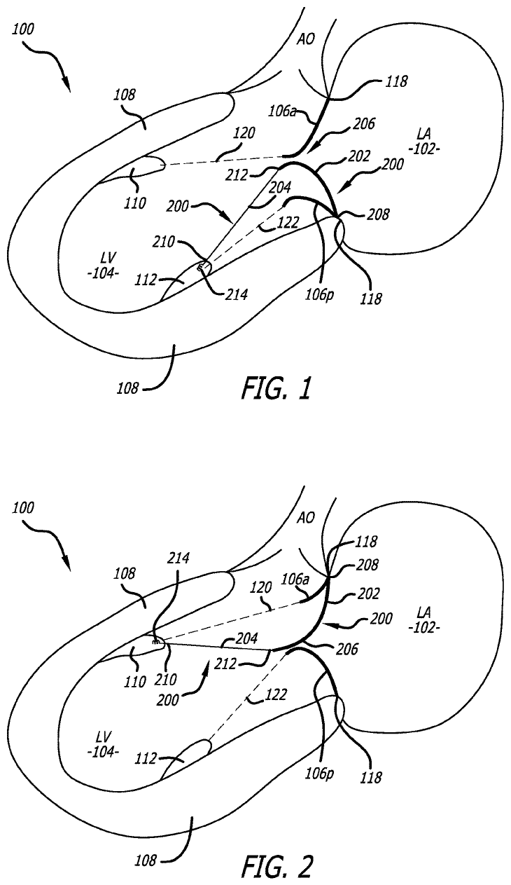

FIG. 1 shows a cross-section of heart 100 with left atrium 102, left ventricle 104, and a native mitral valve between the left atrium 102 and the left ventricle 104. The left ventricle 104 may be defined by a portion of the heart wall 108. The left ventricle 104 has first papillary muscles 110 attached to the heart wall 108 and second papillary muscles 112 attached to the heart wall 108. The native mitral valve has a native anterior leaflet 106a and a native posterior leaflet 106p, which are each connected to a portion of the native mitral annulus, shown generally at 118, which forms a portion of the heart wall 108. The native anterior leaflet 106a is connected to the first papillary muscles 110 with native anterior chord 120. The native posterior leaflet 106p is connected to the second papillary muscles 112 with native posterior chord 122. As shown in FIG. 1, the native posterior leaflet 106p may be diseased, prolapsed, enlarged, or otherwise structurally deformed so as to no longer create a desirable coaptation with the native anterior leaflet 106a, thus resulting in mitral regurgitation.

FIG. 1 shows a leaflet repair device 200 in a fully deployed position to reduce, or in some cases, eliminate the mitral regurgitation caused by at least one of the native mitral valve leaflets. As shown in FIG. 1, the leaflet repair device 200 may be installed without removing the native posterior leaflet 106p or the posterior chord 122. In other embodiments, the native posterior leaflet 106p may be removed or the posterior chord 122 may be cut. As shown in FIG. 1, the leaflet repair device 200 may be installed and fully deployed in a position over the native posterior leaflet 106p and, in some embodiments, the leaflet repair device 200 may overlap the native posterior leaflet 106p. The leaflet repair devices of the present invention may provide improved coaptation of the valve. In some embodiments, the native diseased leaflet may move simultaneously with the leaflet repair device, even though it no longer functions to provide the needed coaptation with the other native leaflet. Moreover, additional leaflet repair devices may be deployed over previously deployed leaflet repair devices to improve coaptation in diseased hearts.

As shown in FIG. 1, leaflet repair device 200 comprises at least an implantable leaflet 202 and a repair chord 204. The implantable leaflet 202 may have a perimeter that is defined by a coaptation edge 206 and an annulus edge 208 opposite the coaptation edge. The coaptation edge 206 is intended to abut another native leaflet in a closed position to improve coaptation of the valve 106. In some embodiments, the implantable leaflet 202 may be anchored, attached, or otherwise connected to the heart wall 108 at or substantially near one or more of trigones, the mitral annulus 118, or other portions of the heart wall 108 near the mitral annulus 118. In some embodiments, the implantable leaflet 202 may be anchored with at least two or more anchors (not shown), one positioned at each respective trigone near the mitral annulus 118. The implantable leaflet 202 may have an annulus edge 208 opposite the coaptation edge 206, and the annulus edge 208 may abut the native annulus when fully deployed. In some embodiments, portions of the annulus edge 208 may be sealed against a portion of the native mitral annulus 118. In some embodiments, the implantable leaflet 202 may be desirably shaped to complement, correct, or mimic the native structure of the mitral valve. In some embodiments, the implantable leaflet 202 may be a custom shaped leaflet, manufactured to particular dimensions and characteristics of the mitral valve of the individual patient. In some embodiments, the implantable leaflet 202 may be connected directly to the papillary muscle 112 or the myocardial wall 108 without repair chord 204 as an extended shape of the leaflet.

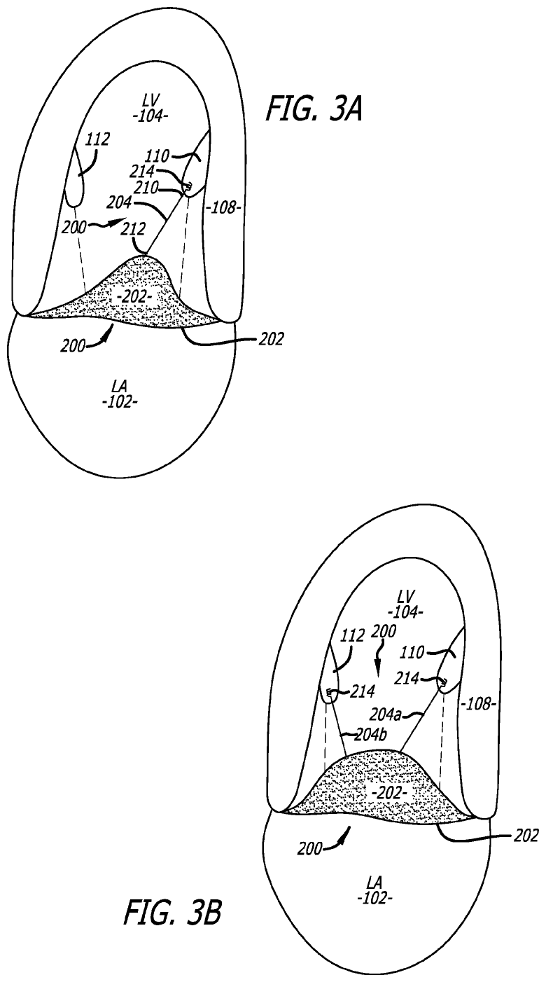

FIG. 2 depicts an implantable leaflet 202 in the same manner as in FIG. 1, but with respect to addressing a diseased anterior leaflet 106a instead of a diseased posterior leaflet 106p. In all other material respects, the above discussion with respect to FIG. 1 applies to FIG. 2.

The implantable leaflet 202 comprises a tissue or synthetic material. The implantable leaflet 202 may be constructed, in some embodiments, from a single piece of tissue material. In other embodiments, the implantable leaflet 202 may be constructed from multiple pieces of tissue material. In some embodiments, the tissue material may be a biomaterial. In some embodiments, the tissue material may be a cross-linked collagen based-biomaterial that comprises acellular or cellular tissue selected from the group consisting of cardiovascular tissue, heart tissue, heart valve, aortic roots, aortic wall, aortic leaflets, pericardial tissue, connective tissue, dura mater, dermal tissue, vascular tissue, cartilage, pericardium, ligament, tendon, blood vessels, umbilical tissue, bone tissue, fasciae, and submucosal tissue and skin. In some embodiments, the tissue material is an implantable biomaterial such as the biomaterial described in the disclosure of U.S. Pat. No. 9,205,172, filed on Dec. 21, 2005 and entitled “Implantable Biomaterial and Method of Producing Same,” which is incorporated by reference herein in its entirety. In some embodiments, the tissue material may be the ADAPT® material manufactured by Admedus Limited. In some embodiments, the tissue material may be selected based on calcification resistance of the tissue material and durability of the tissue material. In some embodiments, the tissue material may be artificial tissue. In some embodiments, the artificial tissue may comprise a single piece molded or formed polymer. In some embodiments, the artificial tissue may comprise polytetrafluoroethylene, polyethylene terephthalate, other polymers, and other polymer coatings. In some embodiments, the artificial tissue may be combined with fabrics or other coatings to encourage cellular growth. The leaflet 202 may be constructed out of any of these materials, either alone or in combination. The leaflet 202 may have coatings, fabric, or other materials embedded in to the leaflet for improved properties.

One or more repair chords 204 may be connected to the implantable leaflet 202 to assist with movement of the leaflet and provide tension onto the leaflet. The repair chord may comprise artificial materials, including but not limited to wires, metallic, ceramics, plastics, fabrics, fibrous materials, polymers, elastomers and materials with suitable elastic properties. In some embodiments, the chord may comprise a biomaterial which may include but are not limited to collagen, tendons, connective tissue, and other fibers. The repair chord 204 may have a first end 212 and a second end 210 opposite the first end 212. In at least one embodiment, the repair chord 204 may be connected at a first end 212 to the implantable leaflet 202. The repair chord 204 may be connected to the implantable leaflet 202 at or substantially near the coaptation edge 206. In at least one embodiment, the repair chord 204 may be connected at a second end 210 to a portion of the heart wall 108. In at least one embodiment, the repair chord 204 is connected to the portion of the heart wall 108 with at least one anchor 214. More particularly, the repair chord 204 may be connected to the respective papillary muscles, for example at the second papillary muscles 112 as shown in FIG. 1. In an alternative embodiment, the repair chord 204 may be connected at the first end to the implantable leaflet 202 and at the second end to the diseased leaflet, which is still connected to the papillary muscles. Although as shown only one repair chord 204 is used, multiple repair chords may be utilized. The one or more repair chords 204 create a tethered connection between the heart wall 108 and the implantable leaflet 202 to provide tension onto the implantable leaflet 202 and to assist with movement of the implantable leaflet between systole and diastole phases. This discussion regarding repair chord 204 for FIG. 1 applies equally to the embodiment of FIG. 2 which depicts an implantable leaflet that addresses a diseased anterior leaflet 106a.

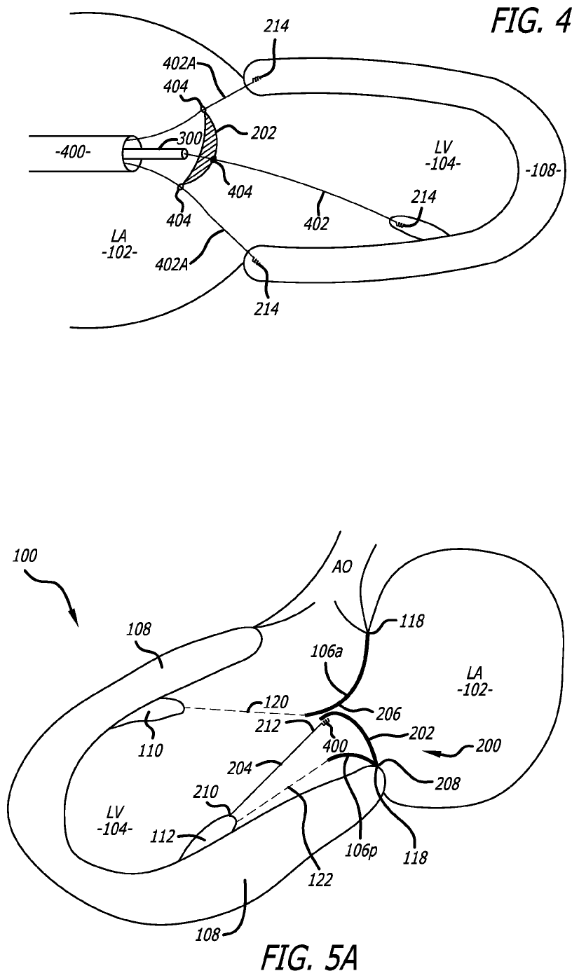

The leaflet repair device of the present disclosure provides flexibility for symmetric and asymmetric placement of the leaflet to address issues in the native valve. FIGS. 3A and 3B show cross-sectional views of the heart. FIG. 3A shows a view of the leaflet repair device 200 with the implantable leaflet 202 tethered with one repair chord 204 to one of the papillary muscles 110, creating an asymmetrical profile of the implantable leaflet 202 as may be needed to address certain issues and conditions of the heart. The implantable leaflet 202 extends more over towards the tethered side. FIG. 3B shows a view of the leaflet repair device 200 deployed with the implantable leaflet 202 tethered with a first repair chord 204a tethered to one of the papillary muscles 110 and a second repair chord 204b tethered in a different direction to another portion of the heart, such as papillary muscles 112. The implantable leaflet 202 can be also directly implanted into the myocardial wall 108 in directions from the mitral valve plane that lead to optimization of leaflet coaptation. In the embodiment shown in FIG. 3B, this deployed leaflet repair device 200 creates a more symmetrical profile than the device shown in FIG. 3A. FIG. 3A shows anchoring of the implantable leaflet 202 to one side of the heart, while FIG. 3B shows anchoring in multiple points of the ventricle. In some embodiments, anchoring to both papillary muscles 110 and 112 as well as the myocardial wall 108 is performed.

Embodiments of the leaflet repair device of the present disclosure may be delivered transeptally to the heart with a transcatheter delivery system. An example or such transseptal delivery of tissue to the heart is found in WO 2019/232068 published Dec. 5, 2019 entitled Method and System for Closure of Cardiovascular Apertures, filed May 29, 2019, the entire contents of which is incorporated herein by reference.

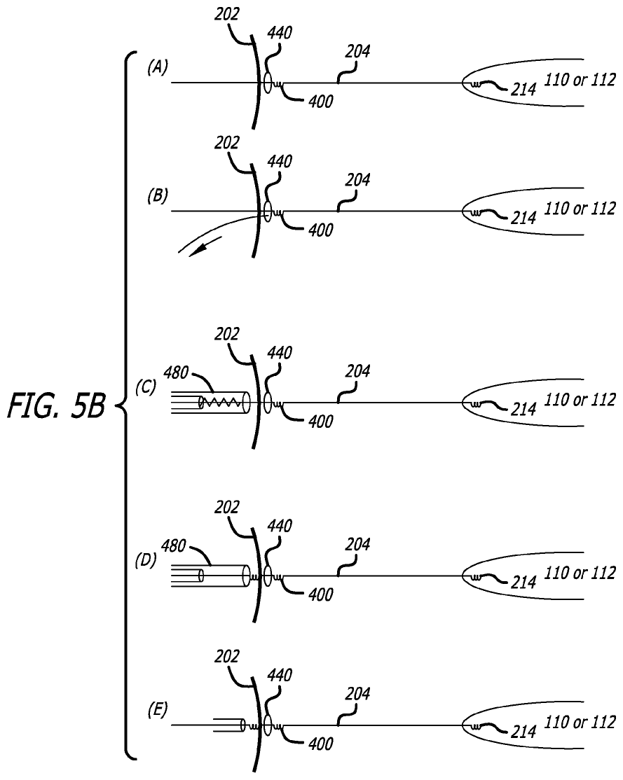

Referring to FIG. 4, the delivery system may comprise a catheter 400, one or more sheaths disposed within the catheter, a pusher catheter 300, an implantable leaflet 202, at least one repair chord 402, and at least one anchoring suture 402A, eyelets 404 and anchoring members 214. In some embodiments, the transcatheter delivery system may further comprise a guidewire (not shown). In at least one embodiment, the catheter may have a distal end and a proximal end. In at least one embodiment of the transcatheter delivery device, in a loaded state, one or more anchoring members 214 are positioned near a proximal end of the catheter. In one embodiment, there is one anchoring member 214 engaged with the repair chord 402 and at least two annulus anchors 214 engaged with sutures 402A. The sutures 402A may be positioned distally from the anchors 214 in the loaded state, and the repair chord 402 may be positioned distally from the anchors 214 in the loaded state. In the loaded state, the implantable leaflet 202 may be in a folded configuration and positioned proximally to the pusher and distally from the sutures 402A and/or repair chord 402.

In one embodiment of the present disclosure, to deliver the leaflet repair device, the anchor members 214 may be deployed first. The chord anchor 402 may be inserted, stapled, sutured, screwed, stitched, plegeted or otherwise fixed to one of the papillary muscles or myocardium. At least one of the annulus anchors 214 may be inserted, stapled, sutured, screwed stitched, plegeted, or otherwise connected to at least one trigone, a portion of the annulus, or the heart wall near the annulus. The anchors may have radiopaque markers or other imaging markers to allow the practitioner to view the position of the anchors on an imaging device.

Once the anchors are positioned, a sheath may then be withdrawn to expose the sutures 402A connected to the annulus anchors 214 and the repair chord 402 connected to the chord anchor 214. In some embodiments, the sutures 402A may be pulled proximally through suture holes in the implantable leaflet, while the implantable leaflet 202 is advanced distally within the catheter. Once the implantable leaflet is advanced within the catheter to the left atrium near the annulus, the implantable leaflet may be unsheathed. The pusher 300 is then used to push the leaflet 202 against each annulus anchor 214 and, for each annulus anchor, the eyelet 404 is then delivered over the anchor 214 to secure the leaflet to the annulus anchor with a locking mechanism (not shown) and the suture 402A may be cut. The pusher 300 is then used to push the leaflet 202 into the left ventricle 104 until the leaflet 202 is desirably oriented to create coaptation in the fully deployed position. Once coaptation occurs, the eyelet 404 is then secured over the chord anchor 402 with a locking mechanism (not shown) and the sutures are cut. The delivery system can then be withdrawn through the vasculature.

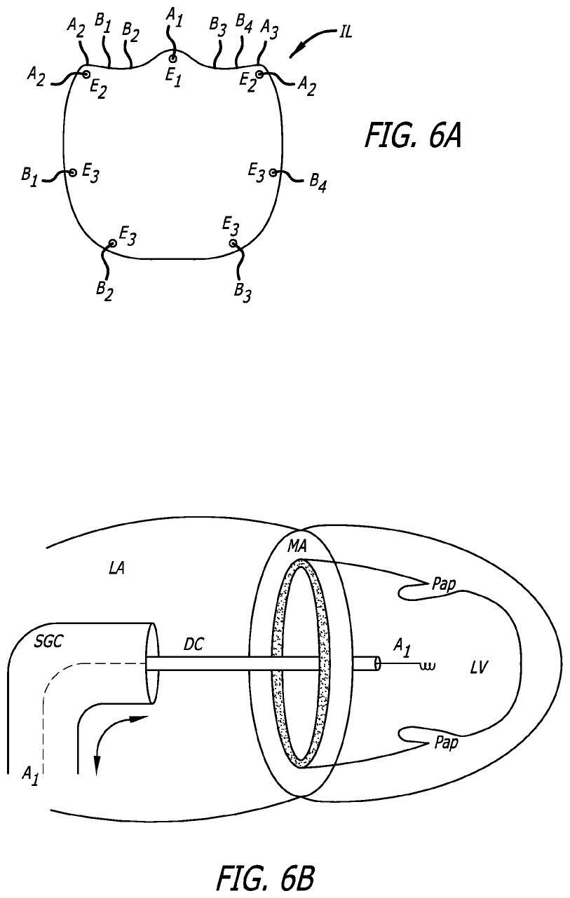

In some embodiments, delivery of the leaflet repair device 200 may require a locking mechanism 400 and a means for trimming the sutures to withdraw the delivery system fully from the patient's body. In at least one embodiment shown in FIGS. 5A-5B, the device may further comprise a locking mechanism positioned in the coaptation zone of the tissue. The locking mechanism may at one end be connected to the repair chord of the device and at the other end may be connected to at least one suture. Tension may be applied to the suture to bias the locking mechanism and pull the locking mechanism from the left ventricle side of the implantable tissue towards through an opening of the implantable tissue to the left atrium side of the implantable tissue. The biased locking mechanism then spans between the left ventricle side of the implantable tissue to the left atrium side.

Mechanisms for use in the present invention include but are not limited to tubular structures that pass over concentrically or coaxially over the element 204 and have members that are biased toward the inner diameter that engage the member 204 such as small barbs that are deformed inwardly from a tubular structure in a manner that when the member 204 is tensioned the barb features engage the material of 204 because of their directional bias and engagement encouraging shape. Another such mechanism could be a stent like structure that is biased to compress onto the member 204 in such a way the delivery mechanism supports the structure with clearance for member 204 and when a desired position is obtained the delivery device releases member 400 and it then reduces in diameter because of resiliently biased construction either entirely or with its barbed members to engage the material of 204 with enough force to maintain its position under clinical loading conditions.

The locking mechanism 400 can be part of the eyelet 440 or a separate mechanism that engages the eyelet 440 and the chord.

In one embodiment, the locking mechanism is similar to a taught line hitch not for securing a stay of a tent or sale and is ideally adjustable in both directions until the user engages the locking mechanism.

In another embodiment, the locking mechanism is slidably free in one direction and locking in the opposite direction. The main object of the locking mechanism is to tighten around and engage the chord member so that the chord member or guide suture does not change in lengthy.

Referring to FIG. 5A, locking mechanism 400 can be made of any suitable, highly elastic and/or shape-memory material such as nitinol. Locking mechanism 400 could also be constructed from a tether material or the same material as the valve, or a polymer, with these materials the locking mechanism would be constructed to operate like a pledget or self-locking knot. This method is well known in the art, the application of the slidable and then lockable knot or pledget would be similar to those used in cardiac surgery.

With reference to FIG. 5B, view a, the chord 204 is attached to the papillary muscle 110, 112 by an anchoring device 214 which is typically known in the art, such as a helical screw-type anchor. The implantable leaflet 202 is introduced over the chord 204 through an eyelet 440 that is present in the implantable leaflet 202.

With reference to FIG. 5B, view b, the tension or distance of the valve tissue 202 via the chord 204 is adjusted and the tension/distance secured by actuating the locking mechanism 400.

With reference to FIG. 5B, view c, a second locking mechanism 400 is introduced via pusher 480 over the chord 204.

With reference to FIG. 5B, view d, the second locking mechanism 400 is urged against the eyelet 440 of the implantable leaflet 202 to further secure the chord 204 to the implantable leaflet 202.

With reference to FIG. 5C, view e, the chord 204 has been cut thus leaving the implantable leaflet 202 with the proper tension/distance from the papillary muscle 110, 112 for proper coaptation with the remaining native leaflet of the mitral valve.

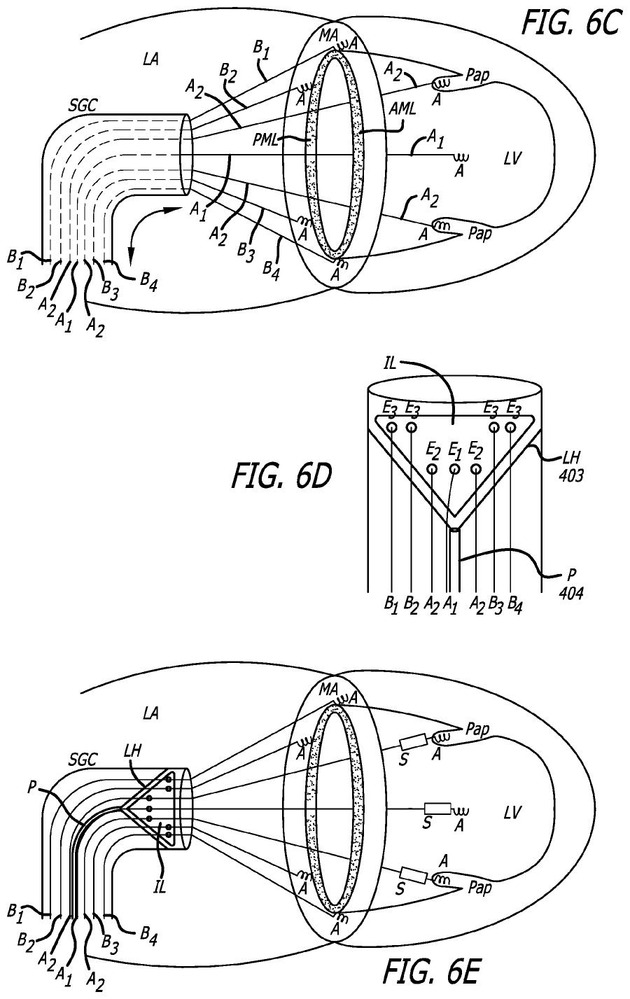

In another embodiment of transseptal delivery of an implantable leaflet in accordance with the present invention, reference is made to FIGS. 6A-6J. Referring first to FIG. 6A, an implantable leaflet IL is shown having center eyelet E1 for receiving a primary chord A1 and two side eyelets E2 for receiving two additional primary chords A2, respectively. Peripheral eyelets E3 are positioned around a peripheral edge of the implantable leaflet IL for use in anchoring the implantable leaflet IL in the annulus of the mitral valve using guiding sutures B1, B2, B3 and B4.

Referring to FIG. 6B, a steerable guide catheter SGC is maneuvered in a manner known in the art to be positioned in the left atrium LA to face the mitral annulus MA. A delivery catheter DC is then extended from the steerable guide catheter SGC so that its distal end extends through the mitral annulus MA into the left ventricle LV. The primary chord A1 having an anchor A at its distal end, is extended into the left ventricle and the anchor is applied to a wall of the left ventricle LV.

Referring to FIG. 6C, the additional primary chords A2, which also have an anchor A at their distal ends, are extended through the mitral annulus MA into the left ventricle LV and each is anchored to a papillary muscle PAP, respectively. The guiding sutures B1, B2, B3 and B4, which also have an anchor A at their distal ends, are extended out of the steerable guide catheter SGC and anchored around the periphery of the mitral annulus. Guide sutures B1 and B4 are located near the conjoining regions of the posterior mitral leaflet PML and the anterior mitral leaflet AML. Guide sutures B2 and B3 are located near the periphery of the posterior mitral leaflet PML.

Referring to FIG. 6D, a view of the implantable leaflet IL is shown being held by a leaflet holder LH inside the steerable guide catheter SGC. The leaflet holder LH extends distally from a pusher P that has been inserted and maneuvered through the steerable guide catheter SGC. The eyelets E1, E2, E3 on the implantable leaflet IL are shown with the primary chord A1, the second primary chords A2 and the guide sutures B1-B4 extending through their respective eyelets.

Referring to FIG. 6E, the configuration of the primary chord A1, the second primary chords A2, the guide sutures B1-B4 in the mitral annulus MA and left ventricle LV is shown just prior to deployment of the implantable leaflet IL from the steerable guide catheter SGC via the pusher P. Note that in this embodiment that spacers S have been placed between the anchor A and the primary chord A1 and the anchors A and second primary chords A2, respectively.

Referring to FIG. 6F, the implantable leaflet IL has been deployed out of the steerable guide catheter SGC and is being urged toward the mitral annulus MA guided primarily by the guide sutures B1, B2, B3 and B4 so that an edge of the implantable leaflet IL mates with the portion of the mitral annulus MA that is adjacent the base of the posterior mitral leaflet PML. The pusher P, which in FIG. 6F has been introduced now to follow guide suture B1, moves a compressible locking screw CLS over guide suture B1 to abut eyelet E3. Referring to FIG. 6G, the guide suture B1 is threaded between at least two coils of the compressible locking screw CLS so that when the compressible locking screw CLS is compressed by the pusher P against the eyelet E3 (when the implantable leaflet is snug against the mitral annulus MA), the guide suture B1 is locked into permanent place in the compressible locking screw CLS. The guide suture B1 is then cut.

Referring to FIGS. 6H, using the pusher P, the user individually urges the implantable leaflet IL over each of the guide sutures B1, B2, B3, B4 against the mitral annulus MA and then urges the coaptation edge of the implantable leaflet IL along the primary chord A1 and the second primary chords A2. The guide sutures B1-B4 and the primary chord A1 and second primary chords A2 are then secured in place against the eyelets using the compressed locking screw CLS associated with each suture/chord and the suture/chord then each being cut. FIG. 6H depicts this process with the mitral annulus MA being in a cross-sectional view so that the entire leaflet can be viewed. FIG. 6I depicts this process with the mitral annulus MA being shown and the implantable leaflet IL being shown in phantom.

Referring to FIG. 6J, the fully implanted and secured implantable leaflet IL is depicted, again with the mitral annulus MA being viewed in cross-section.

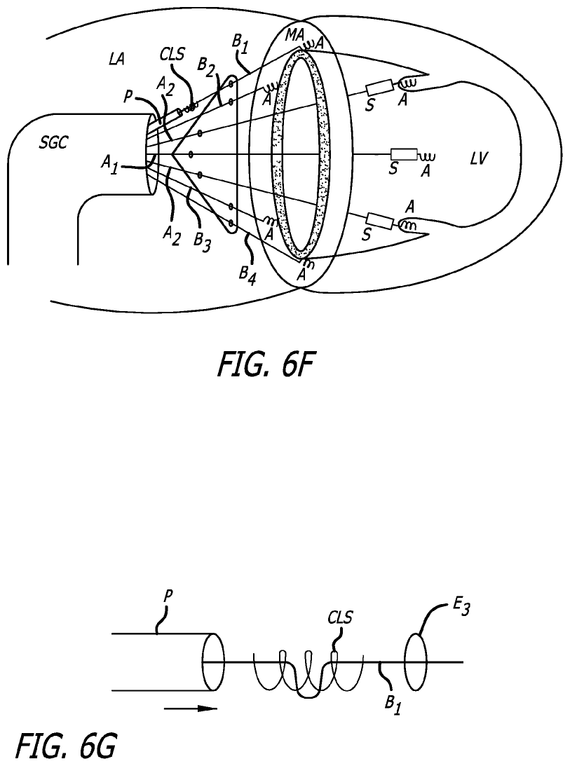

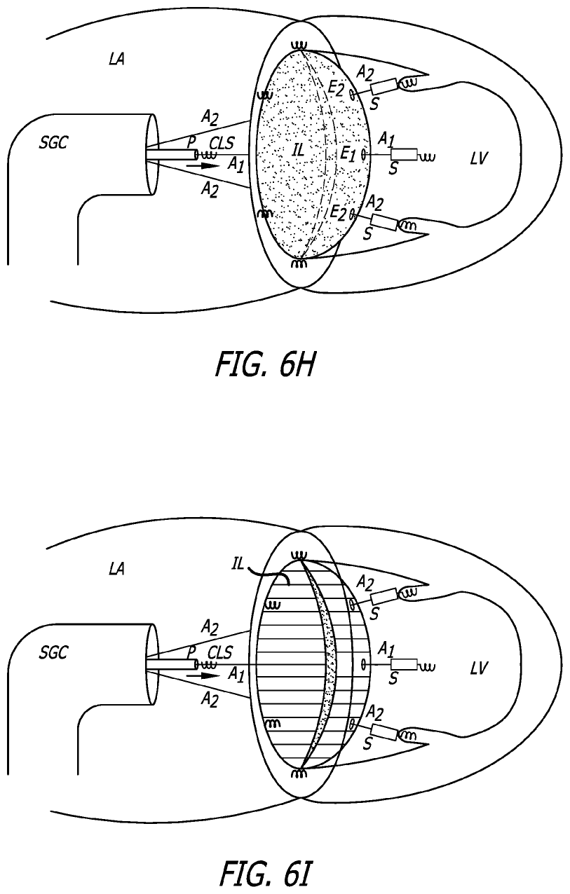

FIGS. 7A-7B show a view of the mitral valve before (A) and after (B) placement of the implantable leaflet 202. In FIG. 7A, there is a large gap from lack of coaptation of the mitral valve leaflets 106a and 106b. In FIG. 7B, anchoring members 214 have been implanted around the mitral annulus with the leaflet anchored inside the left ventricle 204 to create a new coaptation zone.

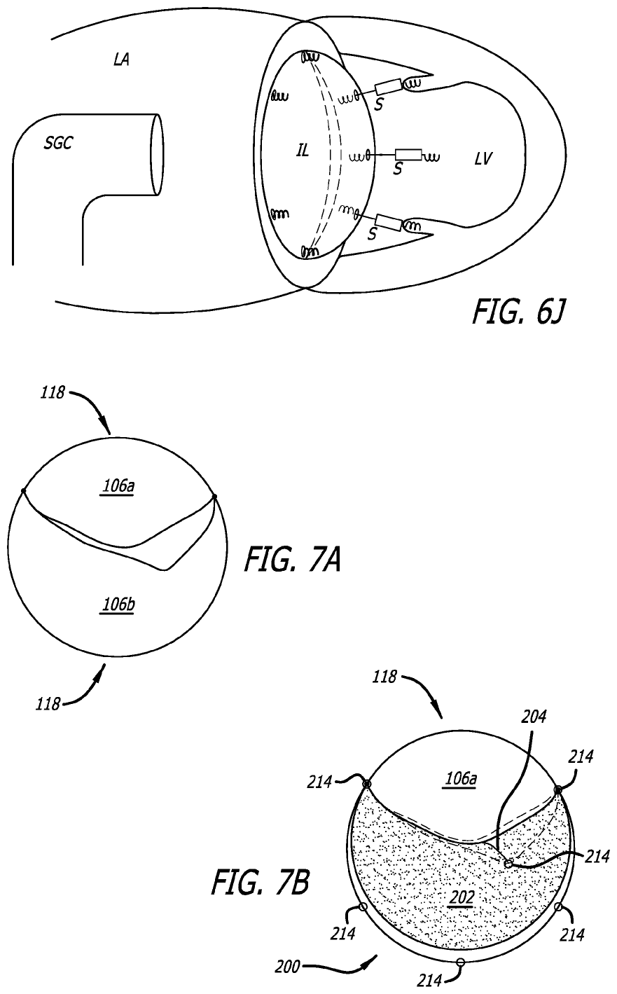

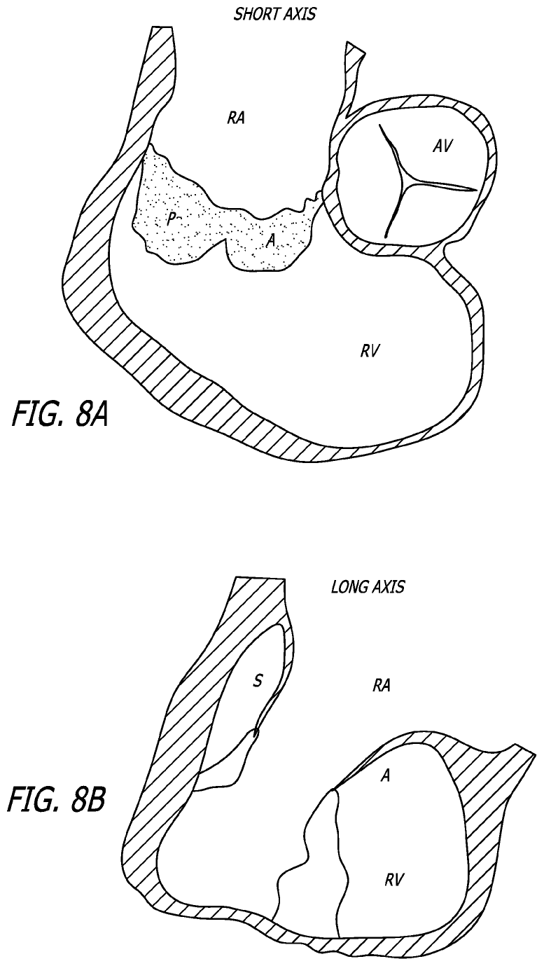

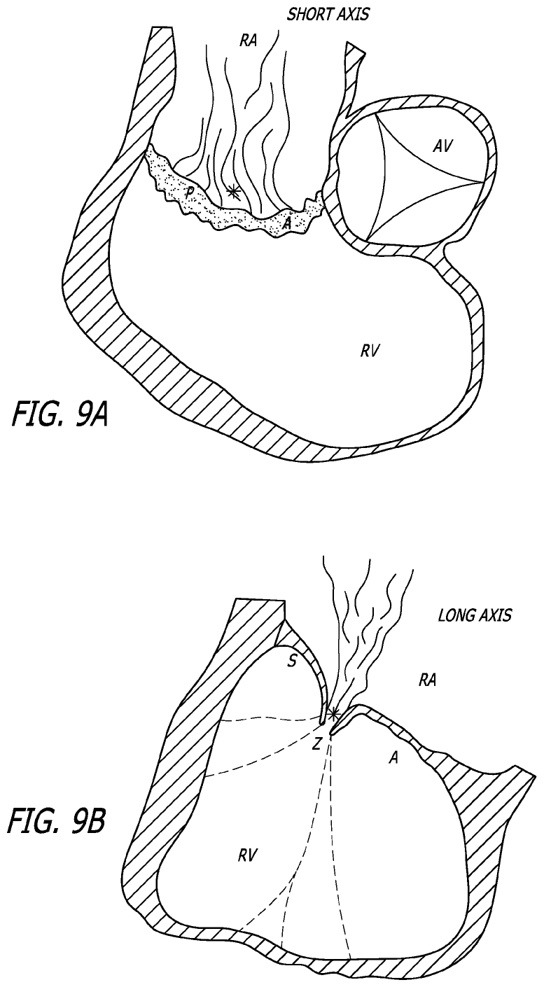

FIGS. 8 to 13 describe use of the implantable leaflet 202 for treatment of a patient with TR. FIGS. 8A, 8B show two cross sectional views of the anterior (A), septal (S), and posterior (P) segments of the tricuspid valve, right atrium (RA), right ventricle (RV), and aortic valve (AV). The tricuspid valve is open in diastole. FIGS. 9A, 9B show two cross sectional views of these same structures in systole, when TR (asterisk) occurs due to loss of leaflet coaptation in the zone of coaptation (Z). FIGS. 10A, 10B show a cross sectional image of the tricuspid valve in diastole and systole. In diastole, the leaflets open normally; however, during systole, regurgitation occurs due to loss of coaptation at the zone (asterisk). For descriptive purposes, CS is the coronary sinus.

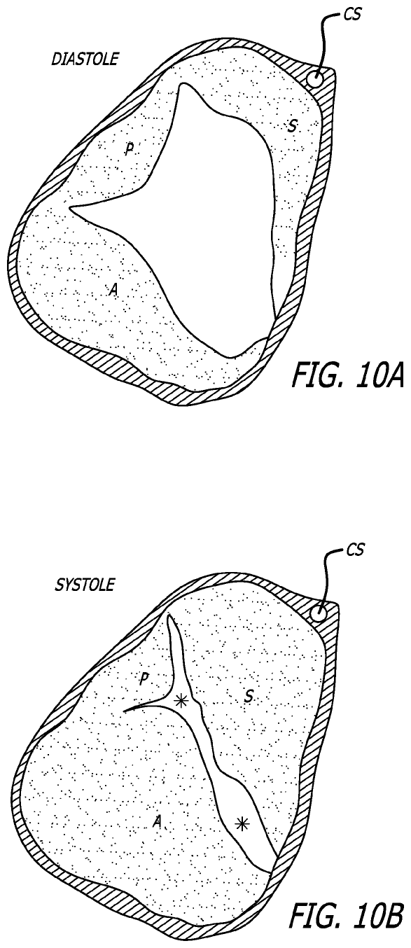

FIGS. 11, 12, and 13 show the tricuspid valve with the implantable leaflet in place. The leaflet (5) is anchored into the right ventricular myocardium with anchoring members (2), supportive members (3) and cords (4). FIG. 11A shows the leaflet from a short axis view of the aorta and right ventricle with multiple anchoring mechanism holding the leaflet during diastole. FIG. 11B shows the leaflet extending over the native septal tricuspid leaflet (S) with opening during diastole.

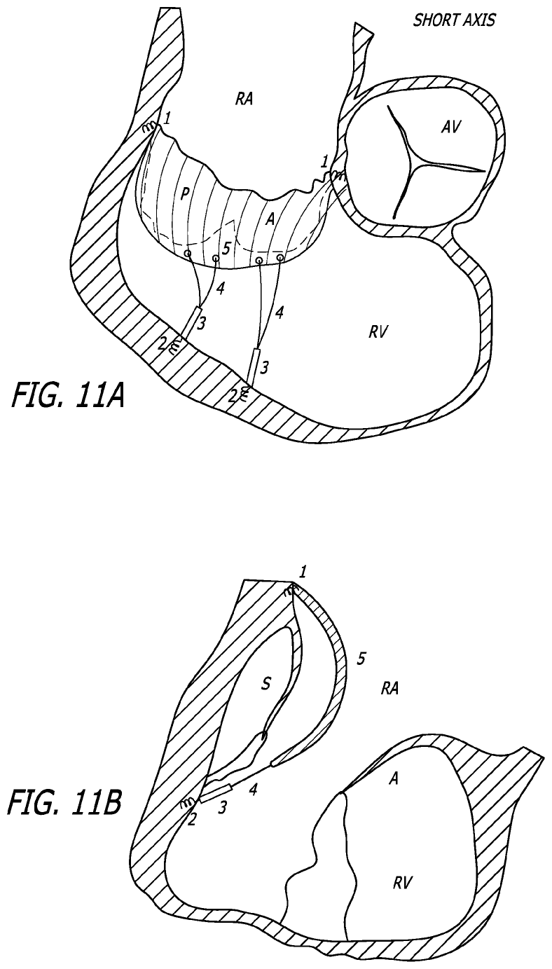

FIG. 12A shows the anchoring and supportive members holding the leaflet in place during systole. FIG. 12B shows the anterior leaflet has closed on the implantable leaflet 5 to create a new zone of coaptation (NZ) and treat TR.

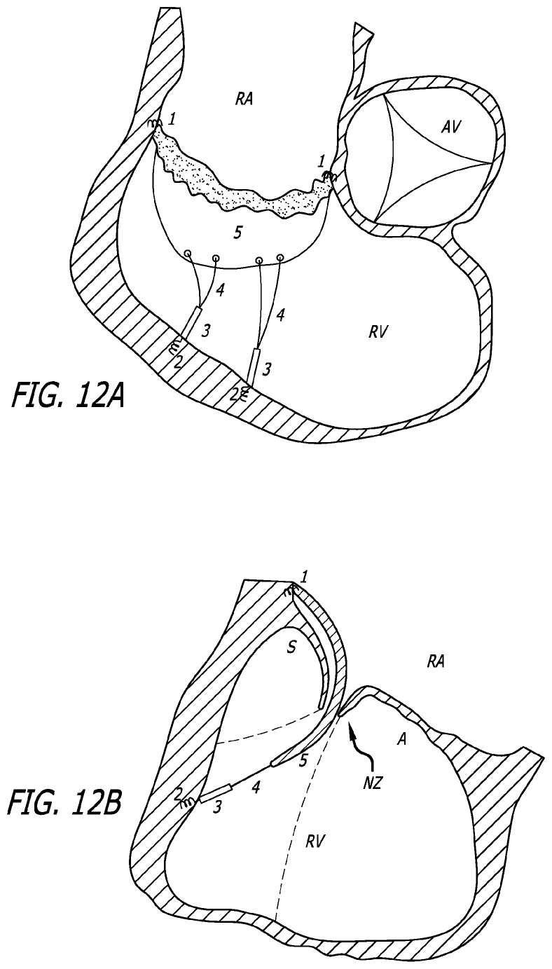

FIGS. 13A and 13B show a cross sectional, short axis view of the tricuspid valve with the implantable leaflet in place. The dotted line shows the old coaptation edge OCE (dashed) of the native septal leaflet as well as the new coaptation line NCL (solid). Referring to FIG. 13A, during diastole, the implantable leaflet moves freely and allows the right ventricle to fill with blood. Referring to FIG. 13B, during systole, the implantable leaflet creates or enhances coaptation by serving as a surface that meets the native anterior and native posterior leaflets. The implantable leaflet is held in place by anchoring members (An) around the tricuspid annulus and connecting to members placed in the right ventricular wall. Similar to its use in the mitral valve, the implantable leaflet may be directly connected to the myocardium with or without cords, and with single or multiple anchors placed in angles to create trajectories that optimize the new zone of leaflet coaptation.

It should be recognized that three leaflets are described in these illustrations and mechanisms of action of the implantable leaflet, as three leaflet configurations are the most common type of tricuspid valve. However, fewer (e.g., two) or more (quadricuspid) tricuspid leaflets can be present in a patient, and these methods are applied in the same approach to create or improve the zone of leaflet coaptation and treat regurgitation.

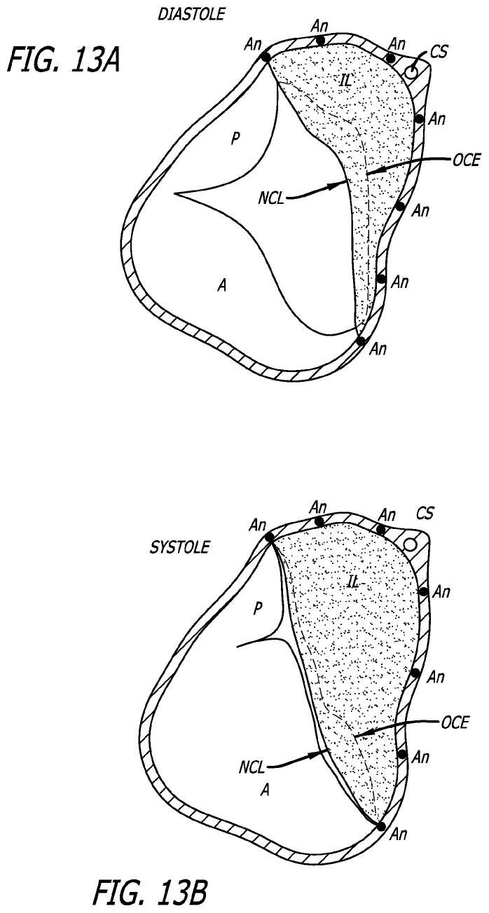

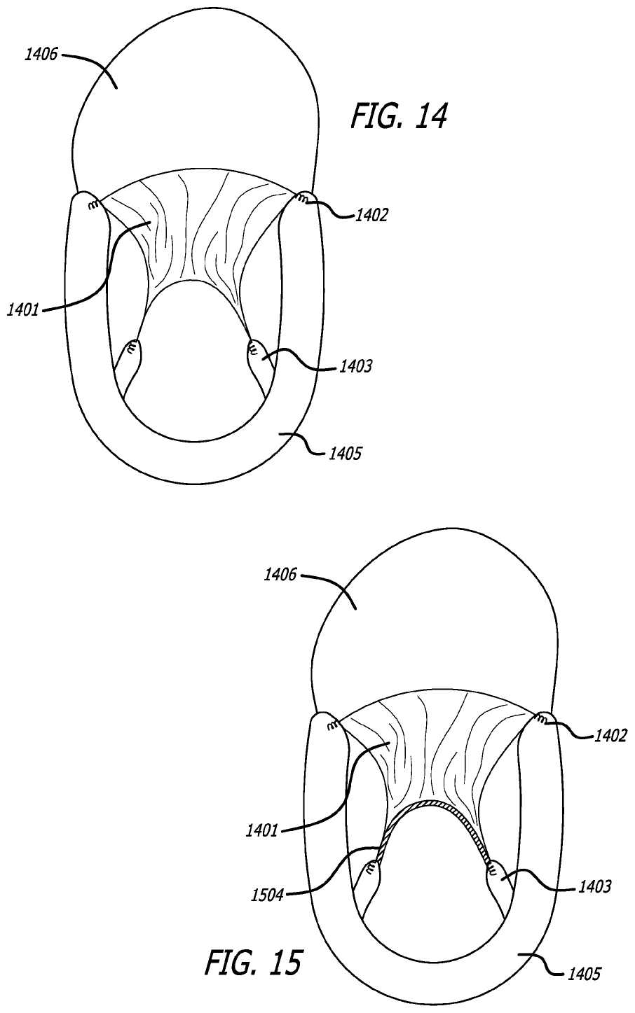

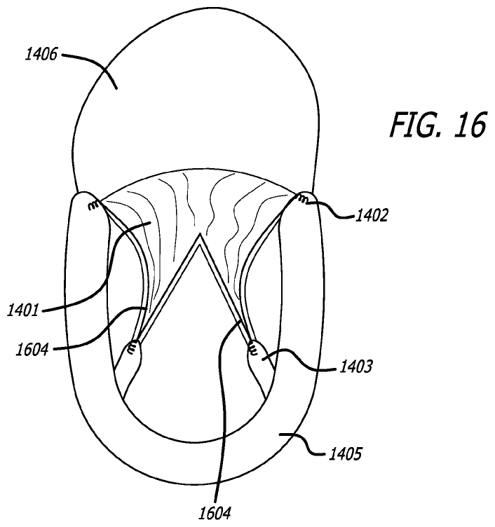

FIGS. 14, 15 and 16 depict another embodiment of the repair device with a unitary construction of a single piece of replacement valve material. The single member constructions could be facilitated with a unitary construction of a single piece of replacement valve material with rolled up edges to reinforce the repair chords of the implantable leaflet. The rolled up material enhances the strength of the replacement valve and facilities smoother transition into the leaflet body. 1401 is a representation of the unitary piece of material shaped to facilitate both the valve and the chordae. 1402 is a representation of the attachment locations along the valve annulus. 1403 is a representation of the attachment locations at the muscle attachment, such as papillary. 1405 is a representation of the ventricular wall. 1406 is a representation of the atrium. FIGS. 15 and 16 are a representation of the cross-sectional views of the repair device with a unitary construction of a single piece of replacement valve material with rolled up edges 1504 (FIG. 15) and 1604 (FIG. 16) to facilitate the repair chord and implantable leaflet.

In one embodiment, the present invention comprises a kit that includes, but is not limited to, a steerable guide catheter, a delivery catheter, a replacement leaflet configured for delivery in and through said delivery catheter, a pusher, a plurality of chords, anchoring mechanisms, and spacers, each of which is disclosed and described herein. The list of contents of the kits is not to be construed as inclusive or exclusive. Some kits may have each of the above items or only some of the above items. Some kits may have more items than listed as needed to undertake the method of the present invention. At a minimum, the kit includes a replacement leaflet and the necessary tools to deliver the replacement leaflet.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an ingredient or element may still actually contain such item as long as there is generally no measurable effect thereof.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description should be read to include one or at least one and the singular also includes the plural unless it is obvious that it is meant otherwise.

Still further, the figures depict preferred embodiments for purposes of illustration only. One skilled in the art will readily recognize from the discussion herein that alternative embodiments of the structures and methods illustrated herein may be employed without departing from the principles described herein.

Upon reading this disclosure, those skilled in the art will appreciate still additional alternative structural and functional designs for the customized urn. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

While the systems and methods described herein have been described in reference to some exemplary embodiments, these embodiments are not limiting and are not necessarily exclusive of each other, and it is contemplated that particular features of various embodiments may be omitted or combined for use with features of other embodiments while remaining within the scope of the invention. Any feature of any embodiment described herein may be used in any embodiment and with any features of any other embodiment.

1. A heart valve repair system comprising:
an implantable leaflet having a coaptation edge and an annulus edge;
a chord for connecting said coaptation edge to a native structure of the heart, said chord having a proximal and a distal end;
a fixation mechanism associated with said chord for attaching said distal end of said chord to said native structure of said heart;
at least one annulus attachment device for securing said annulus edge of said implantable leaflet to an annulus of said heart valve.

2. The heart valve repair system according to claim 1, further comprising multiple chords for connecting said coaptation edge of said implantable leaflet to said native structure of said heart.

3. The heart valve repair system according to claim 1, wherein the implantable leaflet comprises a tissue material.

4. The heart valve repair system according to claim 3, wherein said tissue material comprises a calcification-resistant implantable biomaterial.

5. The heart valve repair system according to claim 3, wherein said tissue material comprises mammal tissue.

6. The heart valve repair system according to claim 3, wherein the tissue material comprises a synthetic material.

7. The heart valve repair system according to claim 1, further comprising a fixation mechanism for attaching a proximal end of said chord to said implantable leaflet.

8. A method for repairing a valve in a heart comprising:
obtaining a replacement leaflet having an annulus edge and a coaptation edge;
delivering said replacement leaflet to a malfunctioning heart valve;
attaching said annulus edge of said replacement leaflet to an annulus of said malfunctioning heart valve;
connecting said coaptation edge of said replacement leaflet to a native structure of said heart.

9. A method according to claim 8, wherein delivering said replacement leaflet comprises delivering said replacement leaflet transeptally

10. A method according to claim 8, wherein repairing a valve includes repairing one of a group of heart valves consisting of a mitral valve, a tricuspid valve, an aortic valve, and a pulmonary valve.

11. A method according to claim 8, wherein the connecting of said coaptation edge of said replacement valve to a native structure of said heart comprises securing a chord between said coaptation edge and said native structure of said heart.

12. A method according to claim 8, wherein said attaching an annulus edge of said replacement leaflet to said valve annulus comprises anchoring said annulus edge to said valve annulus.

13. A method according to claim 8, wherein a native leaflet of said valve is allowed to remain in place.

14. A method according to claim 8, wherein a native leaflet of said valve is excised prior to delivering said replacement leaflet.

15. A method according to claim 8, wherein after delivering said replacement leaflet to said malfunctioning heart valve, a plurality of chords are deployed and anchored in tissue associated with said heart valve.

16. A method according to claim 15, wherein said replacement leaflet is urged along said plurality of chords towards a fixation location in said annulus of said malfunctioning heart valve.

17. A method according to claim 16, wherein an annulus edge of said replacement leaflet is anchored to said annulus of said valve and at least some of the plurality of chords are cut.

18. A method according to claim 17, wherein an anchor chord of said plurality of chords is evaluated for tension between said coaptation edge of said replacement leaflet and said tissue associated with said heart valve.

19. A method according to claim 18, wherein said anchor chord is cut when a target tension has been established.

20. A method according to claim 17, wherein multiple anchor chords are evaluated for tension.

21. A kit for treating a malfunctioning valve of a heart comprising;
a steerable guide catheter;
a delivery catheter;
a replacement valve leaflet configured for delivery in said delivery catheter through said steerable guide catheter;
a plurality of chords for securing said leaflet into tissue associated with said malfunctioning valve;
a plurality of anchor mechanisms associated with said plurality of chords;
a pusher for deploying said chords and deploying said replacement leaflet.

22. A kit according to claim 21, further comprising a cutter for cutting said chords.

23. A kit according to claim 21, further comprising spacers used to connect at least one chord to native tissue associated with said malfunctioning valve.
